(19) United States
(12) Patent Application Publication
(10) Pub. No.: US 2009/0312697 A1
(43) Pub. Date: Dec. 17, 2009

(54) SURGICAL SEAL WITH VARIABLE DIAMETER

(75) Inventor: Michael A. Zemlock, Prospect, CT (US)

Correspondence Address: Tyco Healthcare Group LP, 60 MIDDLETOWN AVENUE, NORTH HAVEN, CT, 06473, US

(73) Assignee: Tyco Healthcare Group LP

(21) Appl. No.: 12/432,928

(22) Filed: Apr. 30, 2009

Related U.S. Application Data
(60) Provisional application No. 61/073,177, filed on Jun. 17, 2008.

(51) Int. Cl. A61B 17/34

(52) U.S. Cl. 604/95.04; 604/167.01

(57) ABSTRACT

A surgical access assembly dimensioned for positioning within a patient's tissue includes a seal member having an internal channel defined therein for the reception of a closure member, and an aperture extending through the seal member that is configured to removably receive a surgical object upon its insertion into the surgical access assembly. The closure member is selectively actuable to transition the seal member from a first condition, in which the aperture includes a first diameter, to a second condition, in which the aperture includes a second diameter. The second diameter is less than the first diameter and substantially approximates an outer dimension of the surgical object such that a substantially fluid-tight seal is formed therewith.

### CROSS REFERENCE TO RELATED APPLICATION

The present application claims the benefit of and priority to U.S. Provisional Application Ser. No. 61/073,177 filed on Jun. 17, 2008, the entire contents of which are incorporated herein by reference.

### BACKGROUND

1. Technical Field

The present disclosure relates to a surgical access assembly which is removably insertable into a patient's tissue. The access assembly includes a seal adapted for the reception of a surgical object and the formation of a substantially fluid-tight seal therewith.

2. Background of the Related Art

Many surgical procedures are performed through access assemblies, e.g., trocar and cannula assemblies. These assemblies incorporate narrow tubes or cannulae percutaneously inserted into a patient's body, through which one or more surgical objects may be introduced and manipulated during the course of the procedure. Generally, such procedures are referred to as “endoscopic”, unless performed on the patient's abdomen, in which case the procedure is referred to as “laparoscopic”. Throughout the present disclosure, the term “minimally invasive” should be understood to encompass both endoscopic and laparoscopic procedures.

Generally, during minimally invasive procedures, prior to the introduction of a surgical object into the patient's body, insufflation gas is used to enlarge the area surrounding the target surgical site to create a larger, more accessible work space. The maintenance of a substantially fluid-tight seal along the central opening of the access device in the presence of the surgical object is therefore desirable to curtail the escape of insufflation gas and the deflation or collapse of the enlarged work space. To this end, surgical access devices generally incorporate a seal through which the surgical object is inserted.

During the course of a minimally invasive procedure, it is often necessary for a clinician to employ and interchange surgical objects of various sizes, e.g., surgical objects that vary in their outer peripheral dimensions. While many varieties of seals are known in the art, there exists a continuing need for a seal that can accommodate a variety of differently-sized surgical objects in substantially sealed relation to thereby maintain the integrity of an insufflated workspace.

### SUMMARY

In one aspect of the present disclosure a surgical access assembly is disclosed. The surgical access assembly includes an access member dimensioned for positioning in a patient's tissue and defining a passageway that extends longitudinally therethrough that is configured to removably receive a surgical object. The surgical access assembly comprises a seal member including an internal channel formed therein, and at least one closure member at least partially disposed within the internal channel. In alternate embodiments, the at least one closure member extends from the seal member through an egress formed in its proximal surface, periphery, or any other suitable location. The seal member has an aperture that extends therethrough, and is selectively adaptable to transition between first and second conditions upon actuation of the at least one closure member. In the first condition, the aperture includes a first diameter that allows for the insertion of a surgical object, and in the second condition, the aperture includes a second smaller diameter that substantially approximates an outer dimension of the surgical object such that a substantially fluid-tight seal is formed therewith.

In one embodiment, the seal member is formed of a material that is at least semi-resilient in nature such that the aperture of the seal member is normally biased towards the first condition. Alternatively, or additionally, the aperture of the seal member may be biased towards the first condition through the incorporation of at least one biasing member. The biasing member may be disposed within the internal channel, and may define a passage therethrough configured to receive the at least one closure member. In one embodiment, the biasing member is a spring.

The surgical access assembly further includes a housing at a proximal end thereof, from which the access member extends distally. The housing may include at least one opening configured to allow the at least one closure member to pass therethrough such that at least a portion of the at least one closure member is disposed externally of the surgical access assembly.

The surgical access assembly may also include attachment structure configured to releasably secure the at least one closure member when the seal member is in the second condition.

In another aspect of the present disclosure, the at least one closure member is secured to a tensioning mechanism. The tensioning mechanism includes a selectively actuable motor, to which the at least one closure member is operatively secured. The motor may be operatively connected to a spool member, which may in turn be secured to the at least one closure member. The motor is adapted to move the spool member through a plurality of positions to thereby wind and unwind the at least one closure member about the spool member such that the tension applied to the at least one closure member may be respectively increased and decreased.

The tensioning mechanism may also include a sensor operably coupled to the housing of the surgical access assembly. The sensor is adapted to detect at least one attribute of the surgical object, including but not being limited to an outer dimension, color, electrical impedance, or magnetic impedance thereof, upon introduction of the surgical object into the surgical access assembly. In response to the detection of the surgical object, the sensor generates a first electrical signal.

These and other features of the seal disclosed herein will become more readily apparent to those skilled in the art from the following detailed description of various embodiments of the present disclosure.

### BRIEF DESCRIPTION OF THE DRAWINGS

Various embodiments of the present disclosure are described herein below with references to the drawings, wherein:

FIG. 1 is a side, schematic view of a surgical access assembly with a seal disposed therein in accordance with the principles of the present disclosure;

FIG. 2A is a top, schematic view of the seal of FIG. 1 shown in a first condition and including a closure member partially disposed within an internal channel formed in the seal, wherein the closure member includes a first end that is fixedly secured within the internal channel and a second end that extends outwardly therefrom;

FIG. 2B is a side, cross-sectional view of the seal of FIG. 2A;

FIG. 2C is a top, perspective view of an alternate embodiment of the seal of FIG. 2A;

FIG. 3 is a top, perspective view of the seal of FIG. 2A shown in a second condition with a surgical object inserted therethrough;

FIG. 4A is a top, schematic view of another embodiment of the seal of FIG. 2A shown in a first condition and including a biasing member disposed within the internal channel;

FIG. 4B is a side, cross-sectional view of the seal of FIG. 4A;

FIG. 4C is a top, schematic view of the seal of FIG. 4A shown in a second condition;

FIG. 4D is a top, schematic view of another embodiment of the seal of FIG. 4A, wherein the biasing member is disposed between the internal channel and an aperture extending through the seal;

FIG. 4E is a top, schematic view of an alternate embodiment of the seal of FIG. 4A, wherein the biasing member is substantially tubular in configuration;

FIG. 4F is a top, schematic view of one embodiment of the seal of FIG. 4E, wherein the biasing member is disposed between the internal channel and a periphery of the seal;

FIG. 5 is a top, schematic view of yet another embodiment of the seal of FIG. 2A, wherein the first and second ends of the closure member each extend outwardly from the seal;

FIG. 6 is a side, schematic view of an alternate embodiment of the surgical access assembly of FIG. 1, further including a tensioning mechanism; and

FIG. 7 is a side, schematic view of the surgical access assembly of FIG. 6.

### DETAILED DESCRIPTION

In the drawings and in the description which follows, in which like references numerals identify similar or identical elements, the term “proximal” will refer to the end of the apparatus which is closest to the clinician, while the term “distal” will refer to the end which is furthest from the clinician, as is traditional and known in the art. Additionally, use of the term “surgical object” herein below should be understood to include any surgical object or instrument that may be employed during the course of surgical procedure, including but not being limited to an obturator, a surgical stapling device, or the like.

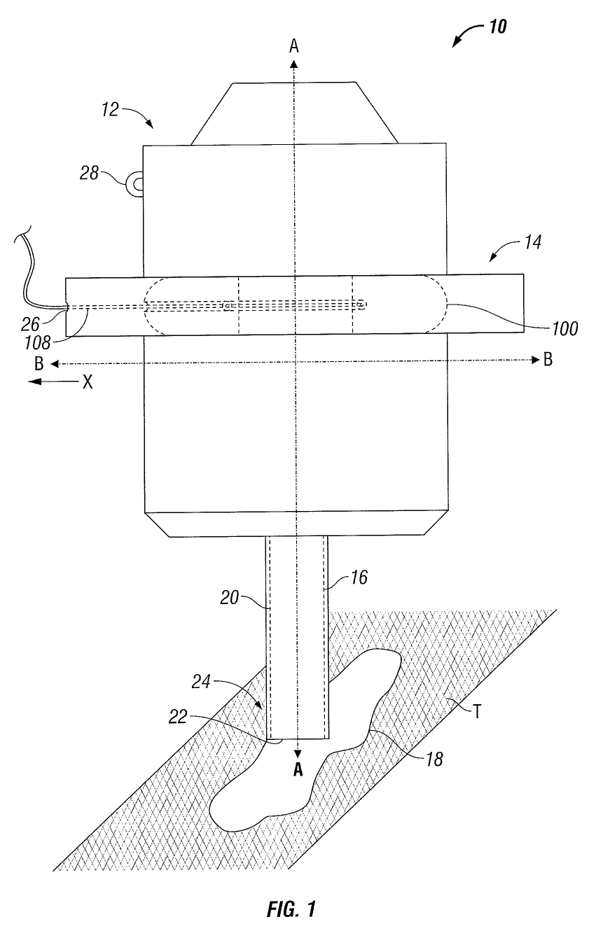

FIG. 1 illustrates a surgical access assembly 10, which is the subject of the present disclosure. At a proximal end 12 thereof, surgical access assembly 10 includes a housing 14 configured to accommodate a seal member 100, and may be any structure suitable for this intended purpose. Further information regarding seal housing 14 may be obtained through reference to commonly owned U.S. Pat. No. 7,169,130 to Exline et al., the entire contents of which are incorporated by reference.

Extending distally from housing 14 is an access member 16 that is dimensioned for positioning with a percutaneous access point 18, either pre-existing or created by a clinician, formed in a patient's tissue “T”. Access member 16 defines a passageway 20 that extends longitudinally therethrough along a longitudinal axis “A-A” and is configured for the internal receipt of one or more surgical objects (not shown). Access member 16 defines an opening 22 at a distal end 24 thereof dimensioned to allow the surgical object (not shown) to pass therethrough, thereby facilitating percutaneous access to a patient's internal cavity with the surgical object.

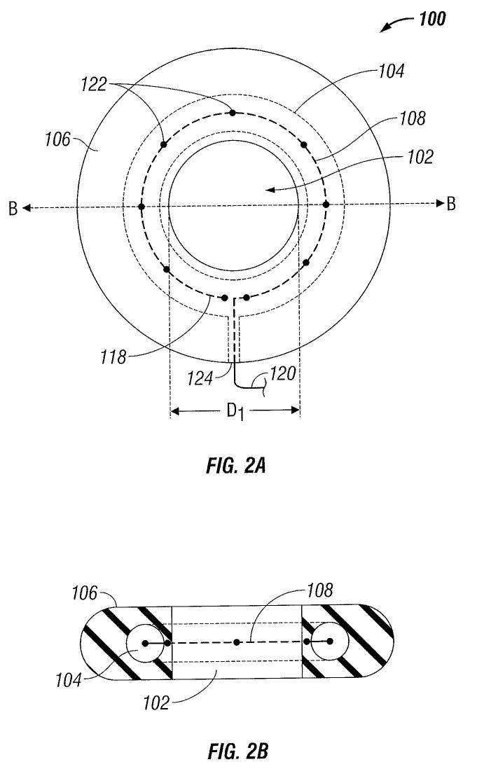

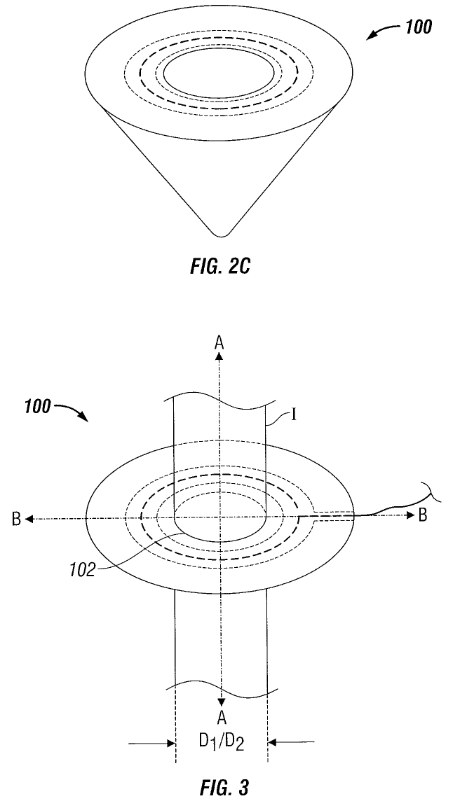

Referring now to FIGS. 2A-5, seal member 100 includes an aperture 102 that extends therethrough and is dimensioned to removably receive the surgical object “I” (FIG. 3) such that a substantially fluid-tight seal may be subsequently formed therewith. In one embodiment, seal member 100 may define a substantially torroidal configuration (FIGS. 2A-2B and FIGS. 3-5), whereas in an alternate embodiment, seal member 100 may define a substantially conical configuration (FIG. 2C) that extends distally to thereby facilitate the insertion of surgical object “I” (FIG. 3) within seal member 100.

Seal member 100 further includes an internal channel 104 formed beneath a proximal surface 106 thereof. Channel 104 is adapted to receive a closure member 108 (or a portion thereof) and is disposed substantially adjacent aperture 102. Channel 104 defines an annular configuration and may be created during the formation of seal member 100, or subsequently thereafter, through any suitable method, including but not being limited to drilling, milling, or molding.

Seal member 100 may be formed of any suitable biocompatible and deformable material, including but not being limited to elastomeric materials such as natural rubber, synthetic polyisoprene, butyl rubber, halogenated butyl rubbers, polybutadiene, styrene-butadiene rubber, nitrile rubber, hydrogenated nitrile rubbers, chloroprene rubber, ethylene propylene rubber, ethylene propylene diene rubber, epichlorohydrin rubber, polyacrylic rubber, silicone rubber, fluorsilicone rubber, fluoroelastomers, perfluoroelastomers, polyether block amides, chlorosulfonated polyethylene, ethylene-vinyl acetate, thermoplastic elastomers, thermoplastic vulcanizers, thermoplastic polyurethane, thermoplastic olefins, resilin, elastin, and polysulfide rubber. Forming seal member 100 from such a material allows the seal member 100 to reversibly transition between a first (unbiased) condition (FIGS. 2A-2B) and a second (constricted) condition (FIG. 3). In the first condition, aperture 102 defines a first dimension “D₁” measured along an axis “B-B” that is transverse in relation to the longitudinal axis “A-A”. The first dimension “D₁” is appreciably larger than an outer dimension “D₁” of surgical object “I” such that surgical object “I” may be inserted into aperture 102 with little resistance. While the use of a surgical object “I” with an outer dimension lying generally within the range of approximately 5 mm to approximately 15 mm is conventional, the employ of substantially larger and smaller surgical objects in connection with surgical access assembly 10 (FIG. 1) is not beyond the scope of the present disclosure.

In the second condition, aperture 102 defines a second transverse dimension “D₂” that substantially approximates the outer diameter “D_{I}” of surgical object “I” such that a substantially fluid-tight seal is formed therewith, thereby curtailing the escape of insufflation gas through seal member 100 when surgical object “I” is inserted therethrough. Forming seal member 100 of a deformable material allows seal member 100, and consequently aperture 102, to substantially conform to the dimensions of surgical object “I” subsequent to the insertion thereof, thereby enhancing the quality of the fluid-tight sealed formed with surgical object “I”.

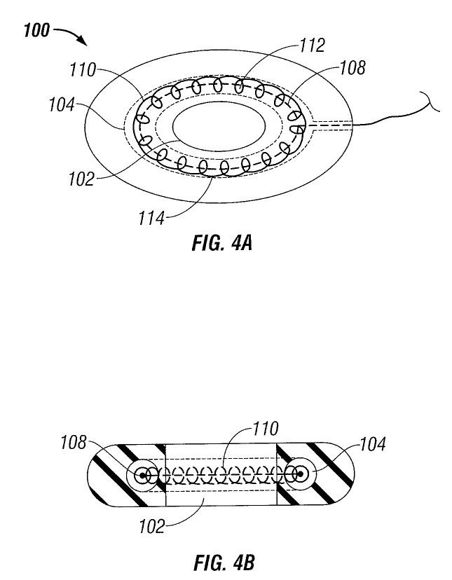

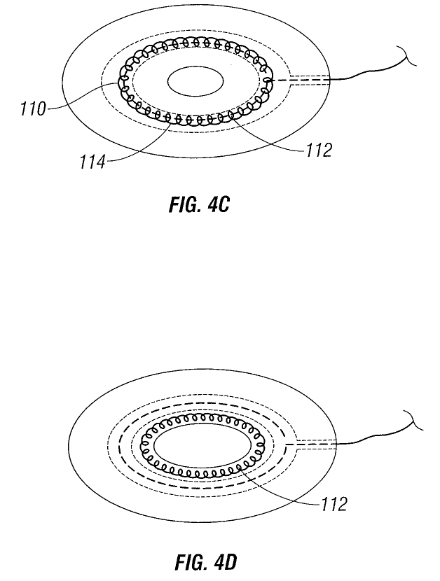

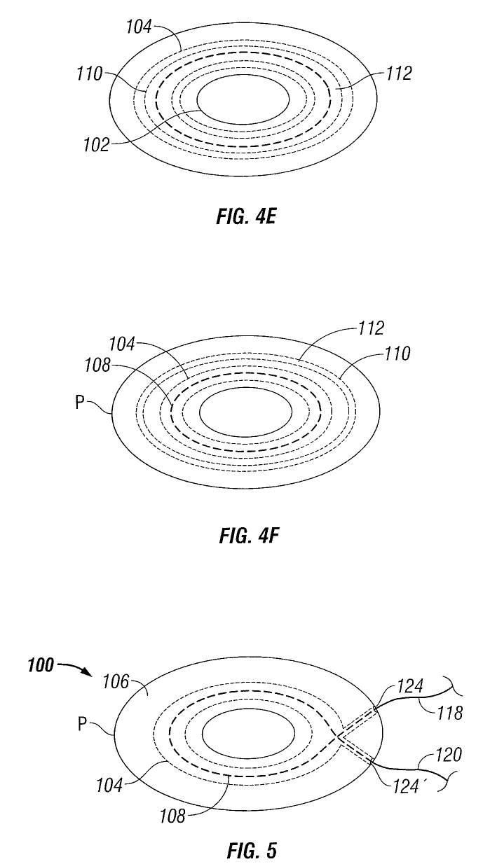

Seal member 100 is adapted to be normally biased towards the first condition. In one embodiment, this is accomplished by forming seal member 100 of a material that is at least semi-resilient in nature. Alternatively, or additionally, the resiliency of seal member 100 may be achieved, or augmented, through the incorporation of one or more biasing members 110. As seen in FIGS. 4A-4F, biasing member 110 defines a passage 112 therethrough which may accommodate closure member 108. Biasing member 110 may be disposed within channel 104 (FIGS. 4A-4C and FIG. 4E), between channel 104 and aperture 102 (FIG. 4D), or between channel 104 and a periphery “P” of seal member 100 (FIG. 4F). Biasing member 110 may be formed of any material adapted to resiliently transition between a normal condition (FIG. 4A) and a deformed condition (FIG. 4C) upon the application of a force thereto and the removal of the force therefrom, respectively. Although biasing member 110 is depicted as a spring 114 in the embodiment of FIGS. 4A-4D, additional adaptations of biasing member 110, such as a hollow tubular structure (FIGS. 4E-4F), are also within the scope of the present disclosure.

Referring now to FIGS. 2A and 5, closure member 108 is an elongated member having respective first and second ends 118, 120. Closure member 108 is at least partially disposed within internal channel 104, as previously described, and acts to facilitate the transition of seal member 100 from the first condition to the second condition, which is discussed herein below. Closure member 108 may be any elongated member, such as a suture, ligature, cable, or the like, formed of a biocompatible material, e.g., stainless steel, fiber, or polymers.

As seen in FIG. 2A, in one embodiment, first end 118 of closure member 108 is secured within seal member 100 at one or more locations 122 within channel 104, whereas second end 120 is free, extending through channel 104, through an egress 124 in communication therewith, and from seal member 100. As seen in FIG. 5, in an alternate embodiment, the respective first and second ends 118, 120 of closure member 108 are each free, extending through channel 104 and from seal member 100 through corresponding egresses 124, 124′ such that the respective first and second ends 118, 120 of closure member 108 cross one another. Egress 124, or egresses 124, 124′, may be formed in any suitable surface of seal member 100, including but not being limited to proximal surface 106 or a periphery “P” thereof. While FIGS. 1-5 depict seal member 100 as including only a single closure member 108, the incorporation of one or more additional closure members is not beyond the scope of the present disclosure.

Referring now to FIGS. 1, 2A-2B, and 3, the use and function of surgical access assembly 10 will be discussed. Initially, the target work site is insufflated with a suitable biocompatible gas, e.g., CO₂ gas, such that a larger internal work space may be created within the patient, thereby providing greater access to internal organs, cavities, tissues, etc. The insufflation may be performed with an insufflation needle or similar device, as is conventional in the art. Following insufflation, access member 16 is positioned within percutaneous access point 18 formed in the patient's tissue “T”, which may be either preexisting or created by the clinician using an obturator (not shown) or the like, as is known in the art. Subsequently, surgical object “I” is introduced to surgical access assembly 10 through housing 14. As seen in FIG. 1, housing 14 includes an opening 26 configured to permit closure member 108 to pass therethrough such that a portion of closure member 108 is disposed externally of surgical access assembly 10. This allows the clinician to apply tension to closure member 108 by applying a force thereto, in the direction of arrow “X” for example, e.g., by grasping and drawing closure member 108 away from surgical access assembly 10.

Prior to the application of any force to closure member 108, seal member 100 is in the first condition (FIGS. 2A-2B) such that surgical object “I” (FIG. 3) may be freely inserted into, and passed through, aperture 102 with relatively little resistance. However, as closure member 108 experiences an increase in tension, seal member 100 transitions from the first condition to the second condition (FIG. 3), in which seal member 100 is in substantial abutment with surgical object “I” such that a substantially fluid-tight seal is formed therewith thereby preventing the escape of insufflation gas through seal member 100. The second condition of seal member 100, and accordingly, the seal with surgical object “I”, may be maintained by sustaining the tension in securing closure member 108. To sustain the tension, closure member 108 may be held, e.g., by the clinician or another party, or closure member 108 may be secured to any suitable surface. In one embodiment, as seen in FIG. 1, surgical access assembly 10 includes attachment structure 28, which may be disposed at any suitable location thereon, including but not being limited to housing 14 or access member 16. Attachment structure 28 is adapted to releasably engage closure member 108, such as by tying closure member 108 thereabout, and may be configured in any manner suitable for this intended purpose.

Following the transition of the seal member 100 from the first condition to the second condition, the remainder of the surgical procedure may then be carried out through surgical access assembly 10. Thereafter, the tension placed on closure member 108 may be relieved, such as by untying closure member 108 from attachment structure 28 in the embodiment of FIG. 1. As the tension on closure member 108 is relieved, seal member 100 returns to its first condition by the bias created through the incorporation of the at least semi-resilient material into seal member 100, the influence of biasing member 110 (FIGS. 4A-4F), or both. During this transition, aperture 102 is enlarged, thereby facilitating the removal of surgical object “I” from seal member 100 will little resistance. Surgical access assembly 10 may then be removed from the access point 18 in the patient's tissue “T”, and the access point 18 may be closed.

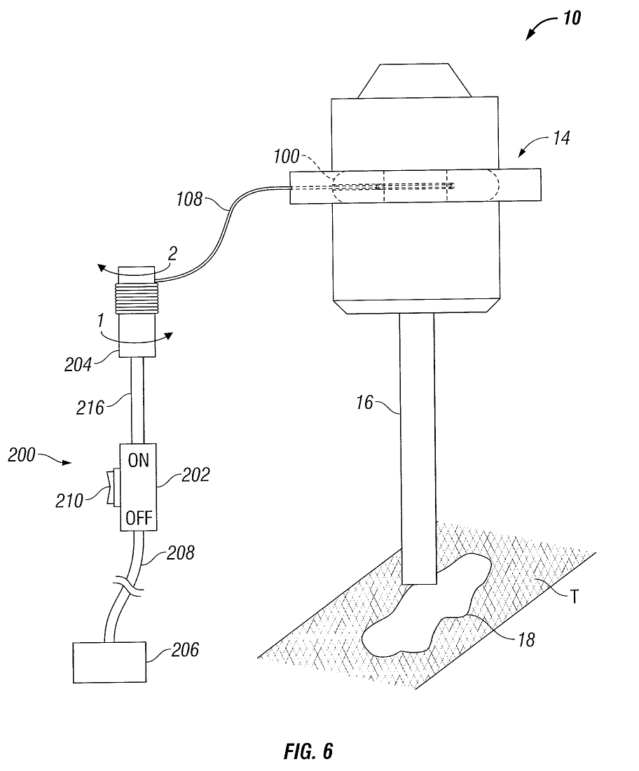

Referring now to FIG. 6, in an alternate aspect of the present disclosure, surgical access assembly 10 may further include a tensioning mechanism 200. Tensioning mechanism 200 may be used to facilitate the transition of seal member 100 from the first condition to the second condition in lieu of manually pulling or drawing upon closure member 108. Tensioning mechanism 200 includes a motor 202 operatively connected to a spool member 204.

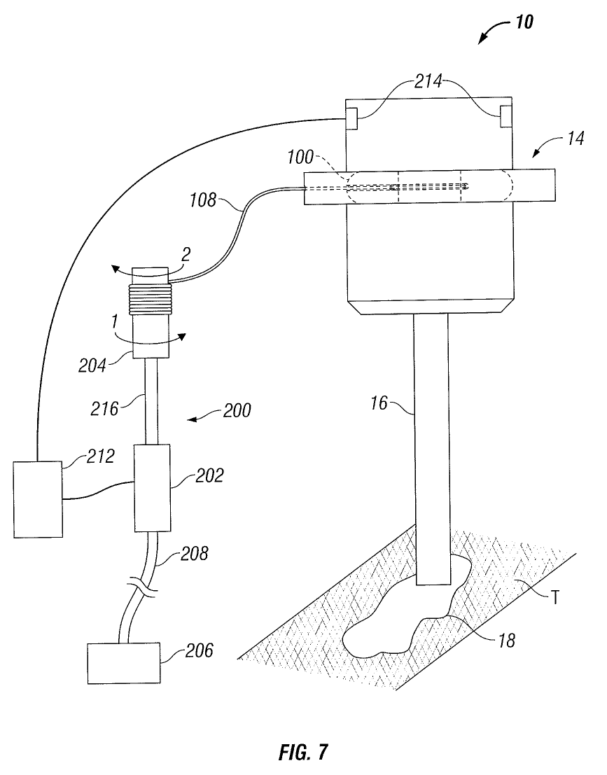

Motor 202 is connected to a power source 206 through a cable or wire 208 such that electrical energy may be communicated thereto, thereby facilitating the selective actuation of motor 202. Motor 202 may be any mechanism suitable for the intended purpose of translating an electrical signal or current into mechanical output. In one embodiment, motor 202 may be actuated by the clinician through the use of an “on/off” mechanism, such as a switch 210. As seen in FIG. 7, in another embodiment, however, the actuation of motor 202 is controlled by a processing unit 212. In this embodiment, processing unit 212 is in communication with motor 202 and sensing means 214, e.g., through the exchange of electrical signals. One or more sensors 214 may be disposed within housing 14, or at any other suitable location within or along surgical access assembly 10, that are responsive to one or more attributes of surgical object “I”, including but not being limited to its color, electrical impedance, magnetic impedance, or outer dimension “D_{I}”, such that sensor 214 may be employed to detect the presence of surgical object “I” upon its introduction to housing 14. Upon detecting surgical object “I”, sensor 214 generates a first electrical signal that is communicated to processing unit 212. Processing unit 212 subsequently interprets the first electrical signal, through the employ of a logic circuit (not shown), and generates a second electrical signal that is communicated to motor 202. The second electrical signal effectuates the actuation, and subsequent control of motor 202, thereby regulating the tension applied to closure member 108, as discussed in further detail herein below.

In the embodiment of FIG. 7, surgical access assembly 10 may further include a delay mechanism (not shown) to provide a specified interval of time between the detection of surgical object “I” by sensor 214 and the subsequent actuation of motor 202.

Motor 202 is connected to a spool member 204 by a shaft 216 such that spool member 204 is moved through a plurality of positions upon the actuation of motor 202. In one embodiment, as seen in FIG. 6, motor 202 is adapted to rotate shaft 216, and correspondingly, spool member 204, in first and second directions, respectively indicated by arrows 1 and 2. Spool member 204 is secured to closure member 108, either releasably or fixed, through any suitable means, including but not being limited to tying closure member 108 about spool member 204 or through the use of an adhesive. Accordingly, as spool member 204 is moved, e.g., in the first direction (FIG. 1), closure member 108 will be wound about spool member 204, thereby increasing the tension in closure member 108 and facilitating the transition of seal member 100 from the first condition to the second condition. Subsequently, or intermittently therewith, closure member 108 may be unwound from spool member 204, thereby decreasing the tension in closure member, e.g., by rotating spool member 204 in the opposite or second direction. As discussed above, seal member 100 is biased towards the first condition, and consequently, decreasing the tension in closure member 108 facilitates the return of seal member 100 to the first condition.

Referring now to FIGS. 2A-2B, 3, and 6-7, the use and function of surgical access assembly 10 in conjunction with tensioning mechanism 200 will be discussed during the course of a minimally invasive procedure. Initially, the target work site is insufflated, access member 16 is positioned within a percutaneous access point 18 formed in the patient's tissue “T”, and surgical object “I” is introduced to surgical access assembly 10 through housing 14, as discussed above with respect to the embodiments of FIGS. 1, 2A-2B, and 3.

Prior to the actuation of tensioning mechanism 200, seal member 100 is in the first condition such that surgical object “I” may be inserted into, and passed through, aperture 102 with relatively little resistance. To transition seal member 100 from the first condition to the second condition, the clinician actuates tensioning mechanism 200. It should be noted that the actuation of tensioning mechanism 200 may occur at any suitable time prior to, subsequent to, or concomitant with the insertion of surgical object “I” into seal member 100.

With respect to the embodiment of FIG. 6, tensioning mechanism 200 may be actuated by engaging switch 210 of motor 202, i.e., by moving switch 210 from the “off” position to the “on” position. Alternatively, with respect to FIG. 7, motor 202 may be automatically actuated subsequent to the detection of surgical object “I” by sensing means 214.

Upon the actuation of tensioning mechanism 200, motor 202 begins to move spool member 204 through its plurality of positions, thereby winding and unwinding closure member 108 about spool member 204 and selectively increasing and decreasing the tension therein to transition seal member 100 to the second condition (FIG. 3) and thereby form a substantially fluid-tight seal with surgical object “I”. The ability to selectively increase and decrease the tension in closure member 108 provides the clinician with a corresponding ability to regulate the second transverse dimension of aperture 102 in the second condition, thereby allowing surgical access assembly 10 to accommodate the use of surgical instruments that vary in size. The tension in closure member 108, and accordingly, the seal formed between seal member 100 and surgical object “I”, may be maintained throughout the duration of the surgical procedure and until such time that closure member 108 is unwound from spool member 204. Thereafter, as the tension in closure member 108 is relieved, seal member 100 returns to its first condition by the bias created through the incorporation of a semi-resilient material into seal member 100, the influence of biasing member 110 (FIGS. 4A-4F), or both. During this transition, aperture 102 is enlarged, thereby facilitating the removal of surgical object “I” from seal member 100 will little resistance. Surgical access assembly 10 may then be removed from the access point 18 in the patient's tissue “T”, and the access point 18 may be closed.

Although the illustrative embodiments of the present disclosure have been described herein with reference to the accompanying drawings, the above description, disclosure, and figures should not be construed as limiting, but merely as exemplifications of particular embodiments. It is to be understood, therefore, that the disclosure is not limited to those precise embodiments, and that various other changes and modifications may be effected therein by one skilled in the art without departing from the scope or spirit of the disclosure.

What is claimed is:

1. A surgical access assembly, which comprises:
an access member dimensioned for positioning with a patient's tissue and defining a passageway extending longitudinally therethrough configured to removably receive a surgical object;
a seal member including an internal channel defined therein and having an aperture extending therethrough, the seal member being selectively adaptable to transition between a first condition wherein the aperture includes a first diameter and a second condition wherein the aperture includes a second diameter that is smaller than the first diameter; and
at least one closure member at least partially disposed within the internal channel which is actuatable to transition the diameter of the seal member from the first condition to the second condition upon actuation thereof.

2. The surgical access assembly of claim 1, wherein the aperture defines a first transverse dimension when the seal member is in the first condition allowing for the insertion of a surgical object and the aperture defines a second transverse dimension when the closure member is actuated to move the seal member to the second condition, the second transverse dimension substantially approximating an outer dimension of the surgical object such that a substantially fluid-tight seal is formed therewith.

3. The surgical access assembly of claim 2, wherein the seal member is formed of a material that is at least semi-resilient such that the aperture of the seal member is normally biased towards the first condition.

4. The surgical access assembly of claim 1, further including at least one biasing member disposed within the seal member, the biasing member being adapted to normally bias the aperture of the seal member towards the first condition.

5. The surgical access assembly of claim 4, wherein the at least one biasing member is disposed within the internal channel.

6. The surgical access assembly of claim 5, wherein the at least one biasing member defines a passage therethrough configured to receive the at least one closure member.

7. The surgical access assembly of claim 6, wherein the biasing member is a spring.

8. The surgical access assembly of claim 1, wherein the at least one closure member extends from the seal member through an egress formed therein.

9. The surgical access assembly of claim 8, wherein the egress is formed in a proximal surface of the seal member.

10. The surgical access assembly of claim 8, wherein the egress is formed in a periphery of the seal member.

11. The surgical access assembly of claim 8, further including a housing at a proximal end thereof, the access member extending distally from the housing.

12. The surgical access assembly of claim 11, wherein the housing includes at least one opening configured to allow the at least one closure member to pass therethrough such that at least a portion of the at least one closure member is disposed externally of the surgical access assembly.

13. The surgical access assembly of claim 12, further including attachment structure configured to releasably secure the at least one closure member when the seal member is in the second condition.

14. The surgical access assembly of claim 12, wherein the at least one closure member is secured to a tensioning mechanism.

15. The surgical access assembly of claim 14, wherein the tensioning mechanism includes a motor operatively secured to the at least one closure member, the motor being selectively actuable.

16. The surgical access assembly of claim 15, wherein the at least one closure member is secured to a spool member operatively connected to the motor, the motor being adapted to reposition the spool member to thereby wind and unwind the at least one closure member about the spool member such that the tension applied to the least one closure member may be respectively increased and decreased.

17. The surgical access assembly of claim 16, wherein the tensioning mechanism includes a sensor operably coupled to the housing, the sensor being adapted to detect at least one attribute of the surgical object upon the introduction thereof into the surgical access assembly and generate a first electrical signal in response thereto to actuate the closure member.

18. The surgical access assembly of claim 17, wherein the at least one attribute of the surgical object is selected from the group consisting of an outer dimension, color, electrical impedance, and magnetic impedance.
